(19) United States
(12) Patent Application Publication
(10) Pub. No.: US 2020/0253307 A1
(43) Pub. Date: Aug. 13, 2020

(54) Perspiration Absorption/Prevention Headband Device with Cooling Means

(71) Applicant: Cindy K. Conner, Fort Wayne, IN (US)

(72) Inventor: Cindy K. Conner, Fort Wayne, IN (US)

(21) Appl. No.: 16/736,897

(22) Filed: Jan. 8, 2020

Related U.S. Application Data
(60) Provisional application No. 62/803,270, filed on Feb. 8, 2019.

(51) Int. Cl. A41D 20/00; A61F 7/02; A41D 13/12

(52) U.S. Cl. CPC A41D 20/005; A61F 7/02; A41D 13/1209; A61F 2007/0007; A41D 2300/326; A61F 2007/0219; A41D 2300/22

(57) ABSTRACT

A perspiration absorption/prevention device designed for use in medical applications, specifically for use by surgeons or other medical personnel. The device is preferably comprised of a headband base member, an absorbent pad, a pocket member and a cooling means stored therein, but may further comprise an adjustment means for further securing the device to a user's forehead or neck area. The cooling means prevents the formation of perspiration on the user, and fatigue generally. Further, any perspiration that does form is quickly absorbed by the absorbent pad before it can drip from the user and onto the patient, surgical tools, etc. The absorbent pad is both detachable from the remainder of the device, and disposable. In this manner, the remainder of the device can be recycled or reused, costs may be lowered and medical waste reduced.

### CROSS-REFERENCE TO RELATED APPLICATION

The present application claims priority to and the benefit of U.S. Provisional Application No. 62/803,270 filed on Feb. 8, 2019, which is incorporated herein by reference in its entirety.

### BACKGROUND

The present invention relates generally to a new and improved headband device for preventing, and also absorbing, a wearer's perspiration. More specifically, the invention relates to a headband device that houses a cooling gel pack and moisture absorbing inner face and is specifically intended for use by surgeons and other medical personnel in a sterile, surgical environment. Accordingly, the present specification makes specific reference thereto. However, it is to be appreciated that aspects of the present invention are also equally amenable to other like applications, devices and methods of manufacture, and that use of the headband device is not specifically limited to a surgical or even medical setting.

Surgeons and other medical professionals routinely perform lengthy and tedious surgeries and other medical procedures, oftentimes in a stressful environment. During said surgeries and/or medical procedures, it is not uncommon for the surgeon or other medical professional to begin to perspire, especially on the surface of their forehead or back of their neck. This can be due to a variety of factors, such as stress related to the complexity of the procedure, operating room temperature, and/or having to wear various medical clothing and/or handle equipment necessary to complete the surgery or other medical procedure.

Perspiration presents a significant risk for contamination in the otherwise sterile surgical environment and to the patient, such as to the patient's open wounds of surgical site. More specifically, with perspiration comes the possibility of bacteria formation, which in turn can contaminate the surgical area and lead to infection for the patient. Specific instances of surgery, such as orthopedic joint replacement, that involve the insertion of a foreign body in the form of an artificial joint already carry a significant risk for infection for the patient. When coupled with the increased probability of bacteria being present due to perspiration of the surgeon or other medical professionals, the likelihood of infection to the patient and a negative outcome only increases.

The current practice/method for preventing perspiration during surgery in the medical field involves a nurse or medical assistant physically and periodically wiping the perspiration from the forehead or neck area of the surgeon with a sterile towel. Not only is this practice/method inefficient in regards to the use of medical personnel, it also still allows for the possibility of contamination to occur due to the unpredictability of the formation and subsequent movement of the perspiration and human error. More specifically, designating a nurse or medical assistant to specifically wipe the forehead or neck area of the surgeon adds another component to their already tedious list of operating room responsibilities. In addition, there will always be a chance that the nurse or medical assistant fails to wipe the perspiration off of the surgeon in time to avoid the perspiration from dripping onto the patient, or worse, the open wound or surgical site, thereby exposing the patent to the surgeon's bodily fluids and the possibility of infection. The nurse or medical assistant's failure to timely wipe the perspiration from the surgeon can be attributable to a number of factors including, but not limited to, inaction, negligence, or the unexpected and rapid formation and/or movement of the perspiration.

Therefore, there exists in the art a long felt need for a perspiration absorption/prevention headband device for use by surgeons and other medical professionals in a surgical setting. There is also a long felt need in the art for a perspiration absorption/prevention headband device that not only absorbs perspiration, but that is also capable of securing a cooling device, such as a gel pack, in close proximity to the surgeon's forehead and/or neck area to prevent perspiration from initially forming, and to keep the surgeon cool and less fatigued. Further, there is also a long felt need in the art for an improved perspiration absorption/prevention headband device that comprises a recyclable portion that is capable of being reused, and that reduces medical expense and surgical waste. Finally, there is a long felt need in the art for an improved perspiration prevention device for use by surgeons and other medical professionals that does not require active involvement from either the surgeon or another individual to use.

The present invention, in one exemplary embodiment, discloses an improved perspiration absorption/prevention headband device comprised of a headband base member, a perspiration absorbent pad, a pocket member, and a cooling means. More specifically, and as explained more fully below, the perspiration absorbent pad may be disposable and is attached to an interior or inner surface of the headband base member so that it is immediately adjacent to the surgeon or other medical professional's skin to absorb any perspiration that emanates therefrom, and to prevent the same from coming into the contact with the patient, surgical tools, etc. Further, the pocket member may be removably attached to an exterior surface of the headband base member and is capable of housing the cooling means, which may be used to cool the skin of the surgeon or other medical professional to prevent the formation of perspiration and reduce fatigue. The cooling means is preferably a reusable gel pack, but may also be any other cooling means known in the art, such as regular ice, dry ice, and the like. While it is contemplated that headband base member will be elastic in nature to conform to the size and shape of the head or neck area of the surgeon or medical professional, the improved perspiration absorption/prevention headband device may further comprise an adjustment system for more securely attaching the device to the head or neck of the wearer.

In this manner, the improved perspiration absorption/prevention headband device of the present invention accomplishes all of the forgoing objectives, thereby improving medical efficiencies and reducing the risk of infection or contamination to the patient. The improved perspiration absorption/prevention headband device of the present invention is also relatively inexpensive to manufacture, and is safe and effective to use. Finally, portions of the improved perspiration absorption/prevention headband device of the present invention may be recycled and/or reused, especially if the need should arise to exchange the gel pack during an extended or lengthy surgical proceeding, thereby reducing expense and the surgical waste stream.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a perspiration absorption/prevention device for use by surgeons and other medical professionals, preferably in a surgical or other medical setting. The perspiration absorption/prevention device is preferably comprised of an elastic mesh headband base member, a perspiration absorbent pad attached to the headband base member and placed adjacent to the wearer's skin to absorb any perspiration emanating therefrom, and a pocket member positioned along or on an exterior surface of the headband base member that is useful for securing a cooling means, such as a reusable or disposable gel back, ice or other cooling mechanism or agent, in proximity to the wearer's skin to prevent perspiration from forming and/or to reduce wearer fatigue.

In a further preferred embodiment of the present invention, the perspiration absorption/prevention device may further comprise an adjustment means, such as elastic or adjustable straps, a buckle, hook and loop fasteners, etc. to more securely attach the device to a wearer's head or neck area, and prevent its unwanted repositioning thereon during movement by the wearer. Generally stated, the perspiration absorption/prevention device significantly reduces the formation of perspiration on the wearer's forehead or neck, and quickly absorbs any perspiration that emanates therefrom and prevent the same from coming into the contact with the patient, surgical tools, etc., which could lead to an infection. Importantly, the device is easy to apply and once secured to the wearer's head, the perspiration absorption/prevention device does not require further intervention from the wearer or other medical personal, such as a surgical nurse and/or medical assistant, thereby enabling said individuals to focus their attention on their other important duties.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a perspective view of one possible embodiment of the perspiration absorption/prevention device of the present invention being worn on the forehead of a user and in accordance with the disclosed architecture.

FIG. 2 illustrates a perspective view of one possible embodiment of the perspiration absorption/prevention device of the present invention being worn on the back of the neck of the user and in accordance with the disclosed architecture.

FIG. 3 illustrates a perspective view of one possible embodiment of the perspiration absorption/prevention device of the present invention being worn on a user's head in accordance with the disclosed architecture and featuring one possible embodiment of a fastening/adjustment system.

FIG. 4 illustrates a perspective view of one possible embodiment of the perspiration absorption/prevention device of the present invention being worn on a user's head in accordance with the disclosed architecture and featuring an alternative embodiment of a fastening/adjustment system.

FIG. 5 illustrates a perspective partial exploded view of one possible embodiment of the perspiration absorption/prevention device of the present invention in accordance with the disclosed architecture.

FIG. 6 illustrates a rear partial perspective view of one possible embodiment of the perspiration absorption/prevention device of the present invention and in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there exists in the art a long felt need for a perspiration absorption/prevention device to aid in both the prevention and absorption of perspiration as it relates to surgeons and other medical professionals, for example, during a surgical procedure. The device of the present invention is designed to both prevent perspiration from forming, for example on a wearer's forehead, neck, etc., and to quickly and efficiently absorb perspiration that does form before it can drop onto a patent, surgical tools, or other sterilized items. More specifically, the device preferably features a headband apparatus having an adsorbent pad removably attached to an interior surface thereof for placement adjacent to the wearer's skin, and a pocket member removably attached to the headband apparatus or integrally formed therewith for housing a cooling means. The cooling means is preferably an exchangeable, reusable or recyclable gel pack, though it is contemplated that other cooling means, such as ice, dry ice, etc. may also be used in other applications, as well as disposable gel packs. The reusable, recyclable and/or disposable gel pack may be placed in a freezer, refrigerator or other cooling station when not in use, and is preferably sterilized prior to use.

The perspiration absorption/prevention device of the present invention is preferably worn about the forehead of a user, with the disposable absorbent pad placed immediately adjacent to the user's skin and the cooling means placed on the opposite side of the headband apparatus from the absorbent pad, but still in proximity to the user's forehead to cool the same. Nonetheless, it is also contemplated that the perspiration absorption/prevention device of the present invention could also be used to prevent and/or absorb perspiration on other portions of the user's body such as his or her neck, arm, leg, etc. When in use, the inboard surface of the absorbent pad absorbs any perspiration from the forehead/neck area of the user, and the gel pad helps cool the same area to prevent further perspiration, and reduce fatigue. The perspiration absorption/prevention device of the present invention also eliminates the need for another individual or the user to physically monitor perspiration, or physically wipe perspiration from their forehead and/or neck area during a medical procedure.

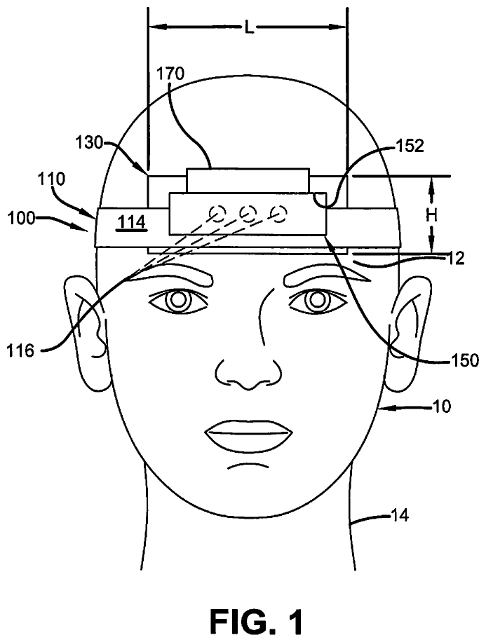

Referring initially to the drawings, FIG. 1 illustrates a perspective view of one potential embodiment of a perspiration absorption/prevention device 100 of the present invention being worn on a forehead 12 of a user or wearer 10. More specifically, the perspiration absorption/prevention device 100 preferably comprises a headband base member 110, an absorbent pad 130, a pocket member 150, and a cooling means 170. While it is contemplated that headband base member 110 will be elastic in nature to conform to the size and shape of the head of the surgeon or medical professional as described more fully below, the improved perspiration absorption/prevention headband device 100 may further comprise an adjustment system for more securely attaching the device 100 to the forehead 12 or neck area 14 of the wearer 10.

More specifically, headband base member 110 is preferably in the form of a continuous loop that can encircle the forehead 12 or neck 14 of a user 10, and comprises both an interior surface 112 and an exterior surface 114. The interior surface 112 faces in the direction of the user's forehead 12 or neck area 14, and the exterior surface 114 faces outwardly from the user 10. Notwithstanding, it is also contemplated that headband base member 110 can be a length of material comprised of two opposing ends that are removably attached to one another to form a continuous loop around a user's forehead 12 or neck area 14, as best shown in the FIGS.

Headband base member 110 is preferably comprised of a mesh material that is both temperature conducive (as explained more fully below) and that exhibits elastic properties that enables it to stretch and retract to accommodate the circumference of a user's forehead 12, and preferably ranges in size from 20 to 27 inches in circumference. Nonetheless, it is also contemplated that the headband base member 110 can come in a variety of different preselected circumferences relating to corresponding head sizes such as, but not limited to, extra small, small, medium, large, or extra-large, and can also be made of elastic banding with hook and loop fasteners.

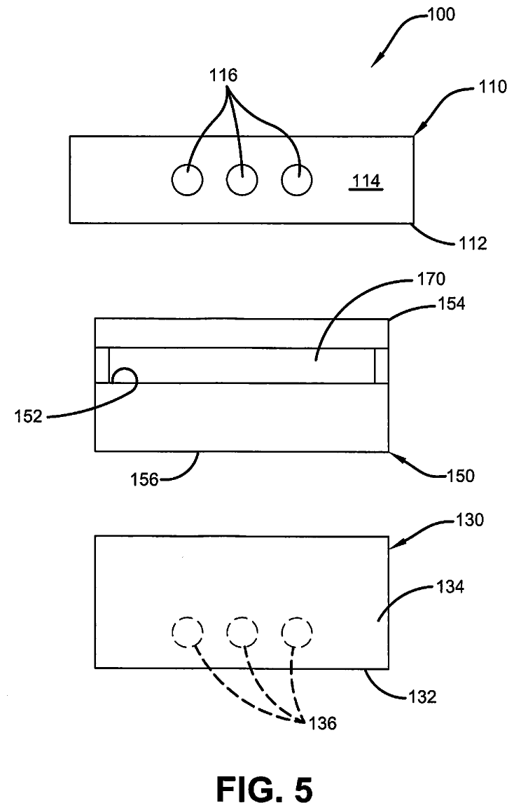

As best shown in FIGS. 1 and 5, headband base member 110 may further comprise one or more fasteners 116 placed on its interior and/or exterior surfaces 112, 114. More specifically, fasteners 116 placed on the interior surface 112 may be used to removably attach absorbent pad 130 to headband base member 110, and fasteners 116 placed on the exterior surface 114 may be used to removably attach pocket member 150 to headband base member 110 such that absorbent pad 130 and pocket member 150 are positioned at roughly the same location along headband base member 110, but on opposite sides thereof. Fasteners 116 may be any type of fastener known in the art (e.g., snaps, buttons, etc.), but are preferably comprised of hook and loop fasteners that matingly engage with corresponding hook and loop fasteners positioned on each of absorbent pad 130 and pocket member 150, as explained more fully below. Inasmuch as perspiration absorption/prevention device 100 is intended to be used in a surgical setting, all components of headband base member 110 are preferably medical grade and sterile.

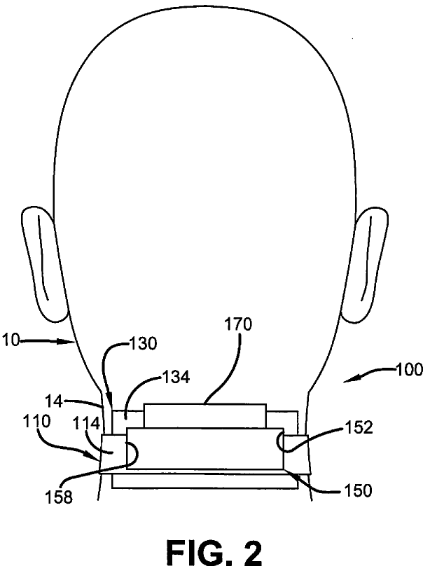

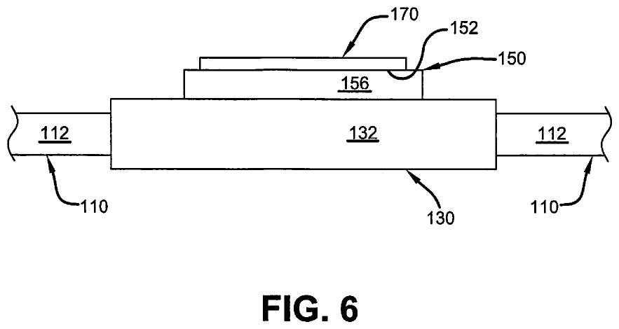

Absorbent pad 130 is preferably generally rectangular in shape and has both a height (h) and a length (l) as best shown in FIG. 1, and is comprised of an inboard surface 132 (as best shown in FIG. 6) and an outboard surface 134. The inboard surface 132 faces the direction of the user's forehead 12 or neck area 14, and the outboard surface 134 faces outwardly from the user 10 in the direction of headband base member 110. Absorbent pad 130 is preferably comprised of an absorbent material, such as medical grade gauze or sponge or those materials found in women's “mini pads” and the like, that is both temperature conducive (as explained more fully below) and that is capable of absorbing any perspiration emanating from the user 10, such as from the forehead 12 or neck area 14, as best shown in FIG. 2. The absorbent pad preferably has a length (l) of 3-5 inches and a height (h) of 2-4 inches to accommodate most user's foreheads 12 or necks 14, although other shapes and sizes of absorbent pad 130 are contemplated as well.

Further, absorbent pad 130 preferably further comprises one or more fasteners 136 placed on its outboard surface 134 for mating engagement with fasteners 116 positioned on the interior surface 112 of headband base member 110. Fasteners 136 may be any type of fastener known in the art (e.g., snaps, buttons, etc.), but are preferably comprised of hook and loop fasteners that matingly engage with corresponding hook and loop fasteners 116 positioned on headband base member 110. In this manner, absorbent pad 130 may be removably attached to the interior surface 112 of headband base member 110 and placed immediately adjacent to the user's forehead 12 or neck area 14 to absorb any perspiration as it emanates from the user's skin. Further, because absorbent pad 130 is removably attached to headband base member 110, it can be discarded after a single use, and the remaining components of perspiration absorption/prevention device 100 may be recycled or reused after proper sterilization, thereby reducing both medical costs and the surgical waste stream.

Alternatively, absorbent pad 130 may be integrally formed with headband base member 110, such that fasteners 116 and 136 may be eliminated. Inasmuch as perspiration absorption/prevention device 100 is intended to be used in a surgical setting, all components of absorbent pad 130 are preferably medical grade and sterile.

Pocket member 150 is also preferably generally rectangular in shape as best shown in FIGS. 1, 2 and 5, and is comprised of an opening 152 therein, a flap 154 for closing said opening 152, and an inboard surface 156. The inboard surface 156 faces the direction of the user's forehead 12 or neck 14, and is adjacent to the exterior surface 114 of headband base member 110, opposite of absorbent pad 130. Pocket member 150 is preferably comprised of a generally waterproof material, such as medical grade plastic, vinyl or the like, that is temperature conducive (as explained more fully below), and the size of the pocket member 150 should correspond to the area of the user's forehead 12 and/or neck area 14 that needs to be cooled. As explained more fully below, the opening 152 of pocket member 150 is for receipt and storage of cooling means 170, and the presence of flap or other enclosure means 154 retains cooling means 170 within pocket member 150 during use.

Pocket member 150 preferably further comprises one or more fasteners 159 placed on its inboard surface 156 for mating engagement with fasteners 116 positioned on the exterior surface 114 of headband base member 110. Fasteners 159 may be any type of fastener known in the art (e.g., snaps, buttons, etc.), but are preferably comprised of hook and loop fasteners that matingly engage with corresponding hook and loop fasteners 116 positioned on headband base member 110. In this manner, pocket member 150 and, by extension, the cooling means 170 removably stored therein, may be removably attached to the exterior surface 114 of headband base member 110.

Alternatively, pocket member 150 may be integrally formed with headband base member 110, such that fasteners 116 and 159 may be eliminated. In a further alternative embodiment, pocket member 150 may comprise a continuous slot or opening 158 in its inboard surface 156 for receipt of a portion of headband base member 110. In this manner, pocket member 150 may slide along the length of headband base member 110, and afford the user 10 with greater flexibility with respect to placement of cooling means 170. Additionally, inasmuch as perspiration absorption/prevention device 100 is intended to be used in a surgical setting, all components of pocket member 150 are preferably comprised of medical grade materials and sterile.

As best shown in FIGS. 1, 2, 5 and 6, cooling means 170 is preferably a gel pack that can be placed in a refrigerator, freezer or other cooling station (not shown) when not in use. Alternatively, cooling means 170 can also be comprised of regular ice, dry ice, or any other item or method commonly known for reducing the temperature of an item or person. Then, as part of the surgery preparations, the cooling means 170 can be placed into the opening 152 of pocket member 150 and enclosed securely therein by flap 154 which may be secured by an additional fastener 159 in a generally waterproof manner (e.g., to prevent water from leaking from pocket member 150 if, for example, cooling means 170 is comprised of regular ice and the same begins to melt). The presence of cooling means 170 in pocket member 150 on the exterior surface 114 of headband base member 110, combined with the temperature conducive nature of pocket member 150, headband base member 110 and absorbent pad 130 described above, provides a cooling sensation to the forehead 12 or neck area 14 of the user 10 and prevents perspiration from forming thereon and fatigue in general. Additionally, inasmuch as perspiration absorption/prevention device 100 is intended to be used in a surgical setting, cooling means 170 (when not regular ice) is preferably comprised of medical grade materials and sterile.

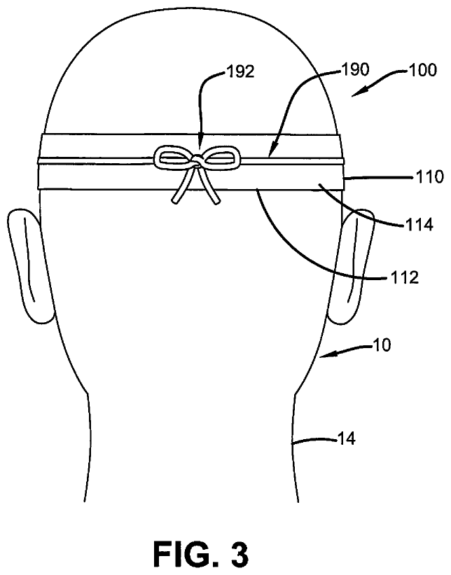

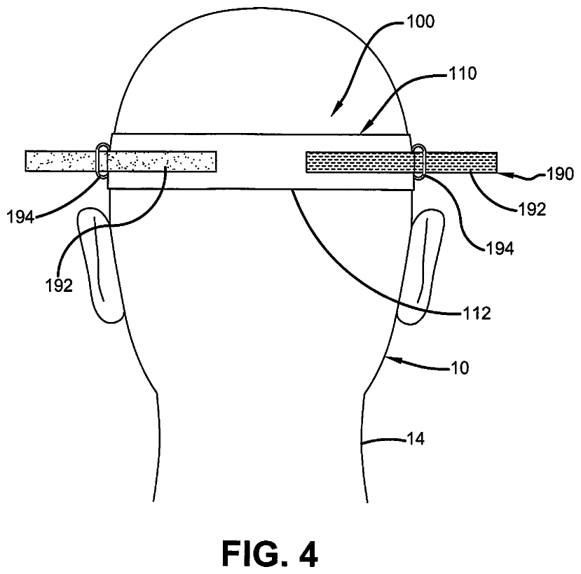

As previously mentioned, while it is contemplated that headband base member 110 will be elastic in nature to conform to the size and shape of the forehead 12 or neck area 14 of the surgeon or medical professional, the improved perspiration absorption/prevention headband device 100 may further comprise an adjustment means or system 190 for more securely attaching the device 100 to the forehead 12 or neck 14 of the wearer 10. Adjustment means 190 may be present in many forms, but is preferably a pair of adjustable straps 192 and corresponding buckles 194 that can be used to increase or decrease the circumference of headband base member 110, as best shown in FIG. 4. Alternatively, buckles 194 may be eliminated, and adjustable straps 192 may simply be tied together as best shown in FIG. 3. Nonetheless, it is contemplated that other adjustment means 190 may also be used, such as elastic straps and the like. Additionally, inasmuch as perspiration absorption/prevention device 100 is intended to be used in a surgical setting, all components of adjustment means 190 are preferably comprised of medical grade materials and sterile.

Importantly, once secured to the wearer's forehead 12 or neck area 14, the perspiration absorption/prevention device 100 does not require further intervention from the wearer 10 or other medical personal, such as a surgical nurse and/or medical assistant, thereby enabling said individuals to focus their attention on their other important duties. Further, notwithstanding the forgoing, the improved perspiration absorption/prevention device 100 can be any suitable size, shape, and configuration as is known in the art without affecting the overall concept of the invention, provided that it accomplishes the above stated objectives. One of ordinary skill in the art will appreciate that the shape and size of device 100 and its various components, as shown in the FIGS. are for illustrative purposes only, and that many other shapes and sizes of the device 100 are well within the scope of the present disclosure. Although dimensions of the device 100 and its components (i.e., length, width, and height) are important design parameters for good performance, the device 100 and its various components may be any shape or size that ensures optimal performance during use and/or that suits user need and/or preference.

It is also contemplated that the device of the present invention may be used to treat, for example, migraine headaches or to apply heat or coolness to an injured portion of the body without the need to continuously hold the heat or cold source adjacent to the body. The device also eliminates the need for mesh or surgical tape to hold a cold compress in place, which could cause irritation to the skin. Further, the gel pack does not require further casing or packaging materials beyond what is typically supplied by the manufacturer, which could unduly increase the thickness and/or weight of the gel-pack, thereby making its use impractical.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A headband device comprising:
a headband base member;
an absorbent pad;
a pocket member; and
a cooling means.

2. The headband device of claim 1, wherein the headband base member is elastic.

3. The headband device of claim 1, wherein the cooling means is a gel pack and is removably housed in the pocket member.

4. The headband device of claim 1, wherein the pocket member is waterproof.

5. The headband device of claim 1, wherein the absorbent pad is removably attached to an interior surface of the headband base member.

6. The headband device of claim 1 further comprising an adjustment means for decreasing an interior circumference of the headband base member.

7. The headband device of claim 6, wherein the adjustment means is comprised of one or more adjustable straps.

8. The headband device of claim 6, wherein the adjustment means is comprised of a hook and loop fastener.

9. A cooling headband device for use in a surgical setting and comprising:
a headband base member;
a disposable absorbent pad removably attached to an interior surface of the headband base member;
a pocket member positioned along the headband base member; and
a gel pack.

10. The cooling headband device of claim 9 further comprising an adjustment means for decreasing an interior circumference of the headband base member.

11. The cooling headband device of claim 10, wherein the adjustment means is comprised of one or more adjustable straps.

12. The cooling headband device of claim 10, wherein the adjustment means is comprised of a hook and loop fastener.

13. The cooling headband device of claim 9, wherein the cooling headband device is sterilized.

14. The cooling headband device of claim 9, wherein the headband base member is a continuous loop and elastic.

15. The cooling headband device of claim 9, wherein the pocket member is positioned on an opposite side of the headband base member from the disposable absorbent pad.

16. A cooling headband device comprising:
an elastic, continuous headband base member;
an absorbent pad removably attached to an interior surface of the headband base member;
a pocket member attached to an exterior surface of the headband base member opposite of the absorbent pad;
a cooling means housed within the pocket member; and
an adjustment means for decreasing an interior circumference of the headband base member.

17. The cooling headband device of claim 16, wherein the adjustment means is comprised of one or more adjustable straps.

18. The cooling headband device of claim 16, wherein the adjustment means is comprised of a hook and loop fastener.

19. The cooling headband device of claim 16, wherein the cooling headband device is sterilized.

20. The cooling headband device of claim 9, wherein the cooling means is a reusable gel pack.
